(19) United States
(12) Patent Application Publication
(10) Pub. No.: US 2026/0275265 A1
(43) Pub. Date: Sep. 17, 2026

(54) MONITORING OF GROWTH OF MICROORGANISMS

(71) Applicant: CarbGeM Inc., Tokyo (JP)

(72) Inventors: Makoto TAKETANI, Tokyo (JP); Mitsutaka NAKADA, Tokyo (JP); Kyohei UMEBAYASHI, Tokyo (JP)

(73) Assignee: CarbGeM Inc., Tokyo (JP)

(21) Appl. No.: 19/473,568

(22) PCT Filed: May 23, 2024
(86) PCT No.: PCT/JP2024/019033

§ 371 Date: Oct. 8, 2025

(30) Foreign Application Priority Data
May 25, 2023 (JP) 2023-086138

(51) Int. Cl. C12M 1/34; C12M 1/00; C12M 1/36; G01N 21/17; G06T 7/00

(52) U.S. Cl. CPC C12M 41/36; C12M 41/06; C12M 41/48; G06T 7/0016; G01N 2021/1748; G06T 2207/10056; G06T 2207/30024

(57) ABSTRACT

A method for monitoring the growth of microorganisms. A solid medium (14) is overlaid on an array (11) in which optical sensor elements (12) larger than the microorganisms (16) are deployed when viewed in plan view. Alignment is maintained over time between the two-dimensional coordinates of the optical sensor elements (12) on the array (11), hereinafter referred to as detection coordinates, and the two-dimensional coordinates on the solid culture medium (14), hereinafter referred to as culture medium coordinates. While maintaining alignment, the light intensity reaching the detection coordinates from the opposite side across each culture medium coordinates is detected by each optical sensor element (12) over time. Changes over time in the intensity of visible light at the culture medium coordinates are observed.

### TECHNICAL FIELD

This invention relates to the monitoring of microbial growth.

### BACKGROUND ART

The method of measuring absorbance (OD600) is known as a technique for monitoring the growth of microorganisms, such as bacteria, in liquid or solid media. The method of measuring colony numbers is widely used in research and clinical testing as a technique for monitoring growth in solid media. In this method, microorganisms are spread on a solid medium, and after a certain period of time, the number of colonies that have grown on the solid medium is counted. Various types of colony counters, which automatically measure the number of colonies, are commercially available.

Non-Patent Literature 1 discloses a system that monitors changes in the number of colonies by capturing images over time. Non-Patent Literature 2 discloses the use of deep learning to detect smaller colonies. Non-Patent Literature 3 discloses a method for measuring colony growth by quantifying colony size.

Patent Literature 1 discloses an imaging system consisting of a matrix of microlenses and a matrix of optical sensors. This system enables the visualization of bacteria (paragraph [0174]). Patent Literature 2 discloses a similar microscopic imaging system. By using this microscopic imaging system, bacteria can be observed (paragraph [0060]).

### CITATION LIST

### Patent Literatures

Patent Literature 1: Patent Publication No. 2012-529025
Patent Literature 2: Patent Publication No. 2018-042283 Non Patent Literatures
Non-Patent Literature 1: Microbio μ3d Autoscanner. Product Catalogue [online]. Becton, Dickinson and Company, [retrieved on Jan. 6, 2023]. Retrieved from https://www.bd.com/ja-jp/offerings/capabilities/microbiology-solutions/industrial-microbiology/systems-for-rapid-check-for-industrial-microbiology/microbio-3d-autoscanner
Non-Patent Literature 2: Li et al. Deep Learning-Enabled Detection and Classification of Bacterial Colonies Using a Thin-Film Transistor (TFT) Image Sensor. ACS Photonics, Volume 9, Issue 7, Jun. 30, 2022, pp. 2455-2466 [online], [retrieved on Jan. 5, 2023]. Retrieved from https://doi.org/10.1021/acsphotonics.2c00572 Non-Patent Literature 3: Bar, J., Boumasmoud, M., Kouyos, R. D. et al. Efficient microbial colony growth dynamics quantification with ColTapp, an automated image analysis application. Scientific Reports Volume 10, Article number: 16084, 2020, [retrieved on Jan. 5, 2023], Retrieved from <https://doi.org/10.1038/s41598-020-72979-4>

### SUMMARY OF THE INVENTION

### Technical Problem

With regard to Non-Patent Literatures 1 to 3, by the time a colony on a solid medium expands to a visible size, the growth curve of the microorganism has already passed the logarithmic phase (Log phase, Exponential phase) and entered the linear phase (Linear phase) or saturation phase (Saturation phase). Increasing the resolution or magnification to detect small colonies in the early stages reduces the observable field of view.

Even if microorganisms seeded on a solid medium can be individually captured microscopically, as described in Patent Literatures 1 and 2, it is not possible to determine solely from the images whether the microorganisms are actually growing on the solid medium or whether they remain stationary after seeding.

In one aspect, the present invention provides a means for monitoring microbial growth that is suitable for early prediction of microbial colony formation within a wide observation field. In another aspect, the present invention provides a means for monitoring microbial growth even before visible colonies appear.

### Solution to Problem

<1> A method for monitoring growth of microorganisms, comprising,
   in a state where a solid medium is superimposed on an array in which optical sensor elements larger than microorganisms are deployed when viewed in plan view, maintaining alignment over time between two-dimensional coordinates of the optical sensor elements on the array, hereinafter referred to as detection coordinates, and two-dimensional coordinates on the solid medium, hereinafter referred to as culture medium coordinates,
   detecting while maintaining the alignment, light intensity arriving at the detection coordinates from the opposite side across each the culture medium coordinates by each of the optical sensor elements over time, and
   observing a change over time in intensity of visible light at the culture medium coordinates. The visible light may include at least one of purple, blue, light blue, green, yellow, orange, and red, for example, at least one of yellow, orange, and red, but is not limited to them.
<2> The method according to <1>, comprising predicting microbial growth based on a decrease in intensity of visible light over time.
<3> The method of the above, comprising
   predicting the growth of the microorganisms by determining that a colony will subsequently occur at the culture medium coordinates at a point in time when the optical density measured over time based on intensity of light detected over time exceeds a predetermined threshold value, hereinafter referred to as a prediction time, wherein
   the threshold value is an optical density value indicating that the optical density will reach a predetermined definitive evaluation value with a predetermined probability at any subsequent time at the culture medium coordinates where the optical density did not reach the predetermined definitive evaluation value at the prediction time.
<4> The method according to <3>, comprising calculating an optical density based on the following equation,

$$[ Optical Density ( OD ) ] = Log ( {I}_{0} / I )$$

wherein I is intensity of light detected at each time point at the detection coordinate, and
I₀ is an average of light intensity for a given number of consecutive detections, including first light intensity detected at the detection coordinates, or I₀ is light intensity detected at each time point at reference detection coordinates aligned with two-dimensional coordinates on an extension of a plane of the culture medium coordinates that is known in advance not to grow microorganisms. It is acceptable here if the solid medium is not superimposed on the reference detection coordinates.
<5> The method according to <3>, comprising,
   dispersing an agent on a surface of the solid medium,
   obtaining frames comprising the culture medium coordinates and light intensity on the culture medium coordinates over time at the array,
   extracting a contour of a set of coordinates in the frame for which the optical density does not exceed the threshold value, and
   extracting a diameter of the set in which the contour is determined to be circular.
<6> The method according to <2>, comprising determining that a colony subsequently arises on the culture medium coordinates at a point in time when intensity of detected light over time decreases and separates from intensity of background light, using a statistical background differencing method, wherein
   background light intensity is calculated based on light intensity for the latest predetermined number of consecutive detections, including the most recently detected light intensity, and
   the separation from the background light intensity is calculated based on a predetermined threshold value.
<7> The method according to <1>, comprising predicting microbial growth based on a decrease over time of combined intensity of light detected by a plurality of adjacent optical sensor elements.
<8> The method according to <1>, comprising calculating an optical density measured over time based on light intensity detected over time using the following equation,

$$[ Optical Density ( OD ) ] = Log ( {I}_{0} / I )$$

where I is light intensity detected at each time point at detection coordinates, and
I₀ is an average of light intensity for a given number of consecutive detections, including the first light intensity detected at the detection coordinates, or I₀ is a reference light intensity detected at each time point at reference detection coordinates aligned with two-dimensional coordinates on the extension of the plane of the culture medium coordinates that is known in advance not to grow microorganisms.
<9> The method according to <8>, wherein
   I₀ is light intensity detected at each time point at the reference detection coordinates, and
   the reference detection coordinates do not have the solid medium overlaid on them.
<10> The method according to <8>, wherein the optical density measured over time is calculated based on the sum of light intensity detected by a plurality of adjacent optical sensor elements.
<11> The method according to <1>, wherein
   the solid culture medium contains an indicator that specifically reacts and changes color in response to the activity of microorganisms belonging to a particular biological classification, and
   the visible light is within a wavelength range that is absorbed by the colored indicator.
<12> The method according to <11>, comprising,
   further observing over time, intensity of light within a wavelength range that is not absorbed by the colored indicator, and
   calculating color space coordinates at the culture medium coordinates.
<13> The method according to <12>, wherein
   the solid culture medium contains multiple types of indicators,
   each of the indicators specifically reacts to growth of microorganisms belonging to different biological classifications, with each indicator producing a distinct color, and
   the biological classification of microorganisms at the culture medium coordinates is identified by calculating the color space coordinates at the culture medium coordinates.
<14> The method according to <13>, wherein
   the light reaching the detection coordinates is divided into blue, green, and red light, and
   timing each color of light separately to ensure arrival at the detection coordinates when detected by the optical sensor elements.
<15> The method according to <13>,
   identifying biological classifications using at least one of the following methods, k-means clustering and other clustering-based analysis methods; principal component analysis, multidimensional principal component analysis and other multidimensional data analysis methods; and rule-based analysis methods; and analysis methods using machine learning.
<16> The method according to <1>, wherein a scale of the culture medium coordinates is equal to a scale of the detection coordinates.
<17> The method according to <1>, wherein
   the optical sensor element is larger than the microorganism when viewed in plan view, or
   the optical sensor element comprises a microlens and a photodetector element, wherein the microlens is larger than the microorganism and the photodetector element is smaller than the microorganism when viewed in plan view.
<18> The method according to <1>, wherein, a wavelength of the visible light is within the range of 400 to 700 nm.

The wavelength of the light may be, for example, 410, 420, 430, 440, 450, 460, 470, 480, 490, 500, 510, 520, 530, 540, 550, 560, 570, 580, 590, 600, 610, 620, 630, 640, 650, 660, 670, 680, or 690 nm, but is not limited to these values.
<19> An apparatus for monitoring growth of microorganisms, comprising:
   a stage for placing a container that holds a solid medium;
   an array with arranged optical sensor elements, each of which is larger than the microorganism when viewed in plan view, and which is fixed in position relative to the stage so as to overlap the solid medium;
   a surface light source provided on the opposite side of the array across the stage;
and a recorder configured to receive, in real time, signals of intensity of light from the surface light source detected by the optical sensor elements, and to record intensity of at least one of the visible light colors, such as violet, blue, cyan, green, yellow, orange, and red, and the time the signal was received.
<20> The apparatus according to <19>, wherein
   the stage is a light-transmissive cover fixed to the surface light source, and
   the container is placed on the stage.

### Advantageous Effects of Invention

In one aspect, the present invention provides a means for monitoring the growth of microorganisms, suitable for early prediction of the appearance of microbial colonies in a wide observation field. In another aspect, the present invention provides a means suitable for monitoring the growth of microorganisms even before the appearance of visible colonies.

### BRIEF DESCRIPTION OF DRAWINGS

FIG. 1: Upper: Side view of the apparatus; Lower: Bottom view of the solid medium.

FIG. 2: Temporal changes in colony radius.

FIG. 3: Temporal changes in optical density.

FIG. 4: Distribution of light intensity in the culture medium coordinates system.

FIG. 5: Temporal changes in light intensity.

FIG. 6: Temporal changes in optical density.

FIG. 7: Temporal changes in the accuracy of microbial growth prediction.

FIG. 8: Distribution of light intensity indicating zone of inhibition.

FIG. 9: Cycles of the three primary colors and the coordinates of the colored culture medium.

FIG. 10: Plot in HSL color space.

FIG. 11: Upper: Side view of the apparatus; Lower: Bottom view of the solid medium.

FIG. 12: Bottom view of the solid medium and the population of microorganisms.

### DESCRIPTION OF EMBODIMENTS

### [Monitoring Device]

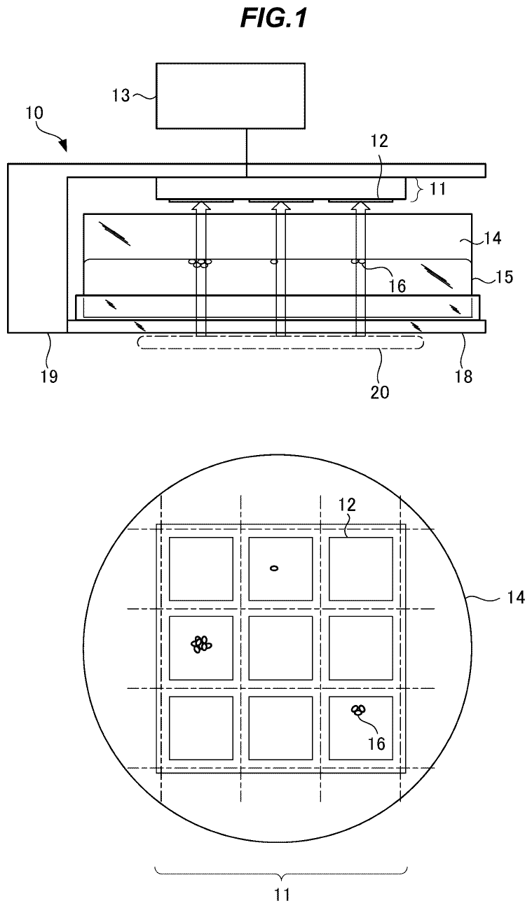

The upper part of FIG. 1 is a side view of a device 10 for monitoring microbial growth. The device 10 includes an array 11, which is equipped with optical sensor elements 12. The device 10 is further equipped with a recorder 13.

As shown in the upper part of FIG. 1, the optical sensor elements 12 are deployed on the array 11. The optical sensor elements 12 are arranged in a two-dimensional matrix on the surface of the array 11. These optical sensor elements 12 may comprise, for example, photodiodes, phototransistors, or photoresistors. The array 11 may be a TFT (thin film transistor) image sensor, a CCD (charge-coupled device) image sensor, or a CMOS (complementary metal-oxide-semiconductor) image sensor.

As shown in the upper part of FIG. 1, a solid medium 14 is layered on top of the array 11. Specifically, the bottom of the container 15 holding the solid medium 14 faces the downward-facing array 11. Microorganisms 16 are seeded on the downward-facing surface of the solid medium 14. However, the colonies of microorganisms 16 may not yet be visible. The solid medium 14 may be, for example, an agar medium. The method of bringing microorganisms 16 into contact with the solid medium 14 is not limited. Airborne bacteria may be dropped onto the sterilized solid medium 14. The solid medium 14 may be stamped onto the surface of the object to be inspected. The method of seeding the solid medium 14 is also not limited. Microorganisms may be smeared using a platinum loop or a Konrad rod. The solid medium 14 may be obtained by mixing a liquid containing microorganisms 16 with a still-liquid medium and then solidifying the mixture. Seeding may also be performed by puncturing the solid medium 14.

The lower part of FIG. 1 shows a bottom view of the array 11, solid medium 14, and microorganisms 16. When viewed in plan view or bottom view, the optical sensor elements 12 are larger than the microorganisms 16. For example, if the microorganisms 16 are E. coli, their length is approximately 1 to 2 μm. The lengthwise and breadthwise length of the optical sensor elements 12 are larger than this. The array 11 constructed with these optical sensor elements 12 does not have the resolution to capture the shape of individual microorganisms. The array 11 depicted in the figure is simplified for illustration purposes. However, the size and pitch of the optical sensor elements 12 may be small enough to acquire images of single colonies of microorganisms 16.

If the optical sensor elements 12 shown in FIG. 1 comprise microlenses and light-receiving elements, the microlenses may be larger than the microorganisms, and the light-receiving elements may be smaller than the microorganisms when viewed in plan view or bottom view.

The size of the optical sensor elements 12 shown in the lower part of FIG. 1 is, for example, larger than 1 μm×1 μm and smaller than 1 mm×1 mm. The lengthwise or breadthwise length of the optical sensor elements 12 may be one of the following values: 2, 5, 10, 20, 30, 40, 50, 60, 70, 80, 90, 100, 110, 120, 130, 140, 150, 160, 170, 180, 190, 200, or 500 μm.

The pitch of the optical sensor elements 12 shown in the lower part of FIG. 1 is, for example, larger than 1 μm×1 μm and smaller than 1 mm×1 mm. The pitch of the optical sensor elements 12, whether lengthwise or breadthwise, may be one of the following values: 2, 5, 10, 20, 30, 40, 50, 60, 70, 80, 90, 100, 110, 120, 130, 140, 150, 160, 170, 180, 190, 200, or 500 μm.

As shown in the lower part of FIG. 1, alignment is maintained over time between the two-dimensional coordinates of the optical sensor elements 12 on the array 11 and the two-dimensional coordinates on the solid medium 14, indicated by the dashed line. The two-dimensional coordinates of the optical sensor elements 12 may hereinafter be referred to as “detection coordinates” while the two-dimensional coordinates on the solid medium 14 may be referred to as “culture medium coordinates”. The culture medium coordinates are virtual. In one aspect, the scale of the culture medium coordinates is equal to the scale of the detection coordinates.

FIG. 1 illustrates that one detection coordinates corresponds to one optical sensor element 12. However, in some cases, a single detection coordinates may correspond to a set of multiple adjacent optical sensor elements 12. For example, the growth of microorganisms may be predicted based on the decrease over time in the combined intensity of light detected by multiple adjacent optical sensor elements 12.

The number of light sensor elements 12 on the array 11, as shown in the lower part of FIG. 1, is not limited. The number of elements in either the lengthwise or breadthwise direction may range from 2 to 20,000. The number of elements in either direction may also be one of the following values: 5, 10, 20, 50, 100, 200, 300, 400, 500, 600, 700, 800, 900, 1000, 1100, 1200, 1300, 1400, 1500, 1600, 1700, 1800, 1900, 2000, 5000, or 10,000.

The size of the array 11, as shown in the lower part of FIG. 1, is determined by the size, pitch, and number of light sensor elements 12. The lengthwise or breadthwise length of the array 11 may range from 2 μm to 1 m in either direction. The length in either the lengthwise or breadthwise direction may also be one of the following values: 1, 2, 5, 10, 20, 30, 40, 50, 60, 70, 80, 90, 100, 110, 120, 130, 140, 150, 160, 170, 180, 190, 200, or 500 mm. When the array 11 and solid medium 14 are viewed in plan view, the array 11 may either fit entirely within the solid medium 14 or extend beyond it.

viewed from the bottom (plan view) of the solid medium 14, as shown in the lower part of FIG. 1, the proportion of the array 11 relative to the solid medium 14 may range from 1% to 100%. The proportion of the array 11 may also be one of the following values: 5%, 10%, 20%, 30%, 40%, 50%, 60%, 70%, 80%, 90%, or 95%.

As shown in the upper part of FIG. 1, the container 15 is placed on the stage 18 with its lid facing downward. Specifically, the container 15 is placed on the stage 18. A petri dish with a lid is illustrated as an example of the container 15 in the figure. The container 15 may also be a multi-well plate or another type of culture vessel.

As shown in the upper part of FIG. 1, the position of the array 11 is fixed relative to the stage 18 so that the array 11 overlaps the solid medium 14. Similarly, the position of the stage 18 is fixed relative to the array 11. This fixation is achieved using a fixing member 19. The alignment between the detection coordinates and the culture medium coordinates is maintained over time by keeping the container 15 stationary on the stage 18.

The solid medium 14, container 15, and stage 18, as shown in the upper part of FIG. 1, are all light transmissive. Light passes through the surface of the solid medium 14 and reaches the array 11 from the opposite side. The device 10 detects the intensity of this light, referred to as luminous flux, at each optical sensor element 12. The device 10 performs this detection while maintaining alignment between the detection coordinates and the culture medium coordinates. Through this process, the device 10 detects the light intensity at each culture medium coordinates over time. Light may pass through the solid medium 14 only when the light intensity is being detected.

As shown in the upper part of FIG. 1, the device 10 may be equipped with a surface light source 20. The surface light source 20 is positioned on the opposite side of the array 11 across the stage 18. In the figure, the surface light source 20 is located at the bottom of the device 10. The surface light source 20 may be turned on only when the array 11 is detecting light intensity.

The stage 18, as shown in the upper part of FIG. 1, may be a light-transmissive cover fixed to the surface light source 20. The stage 18 may serve as a protective cover for the surface light source 20. The stage 18 may serve as a diffusion cover for the surface light source 20. In other configurations, the stage 18 may be a ring-shaped or clip-shaped member that supports the sides of the container 15. The shape of the stage 18 is not limited as long as it does not block the light reaching the array 11 and allows the solid medium 14 to remain stationary relative to the fixing member 19.

The array 11, as shown in the upper part of FIG. 1, is connected to the recorder 13 either directly or indirectly, via wired or wireless communication. In one configuration, signals can be sent and received mutually between the array 11 and the recorder 13. In another configuration, the array 11 may send signals unilaterally to the recorder 13, which then receives them. In one aspect, the recorder 13 receives in real time from the array 11 the signal of the light intensity of the surface light source 20 detected by the optical sensor elements 12. The recorder 13 records both the light intensity and the time the light was received. The recorder 13 may also be a computer.

### <Microorganism Types and Solid Medium Types>

In FIG. 1, microorganism 16 is not restricted as long as it does not migrate on the solid medium. Microorganisms can be bacteria or fungi. Examples of bacteria include Enterobacter cloacae, Escherichia coli (E. coli), and Klebsiella pneumoniae (K. pneumoniae). Microorganisms may be found in humans, non-human animals, or plants. They may also be pathogenic to these organisms. Additionally, microorganisms may be found in environmental sources such as soil, lakes, or oceans.

### <Monitoring Timing>

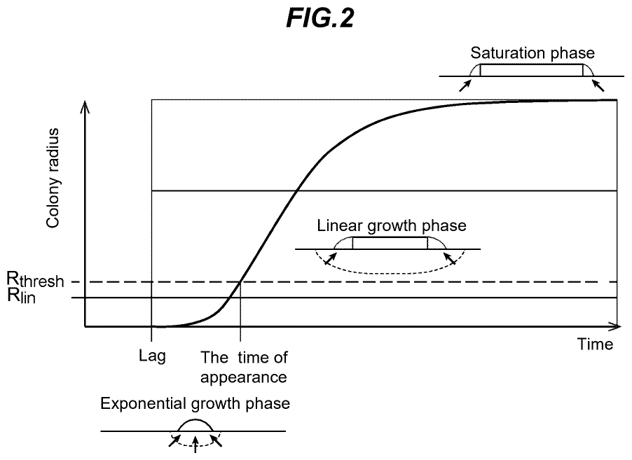

The timing for monitoring microbial growth is explained with reference to the colony growth curve shown in FIG. 2. FIG. 2 illustrates the increase in bacterial colony radius, as cited from Non-Patent Literature 3. Some modifications have been made to improve readability as a patent drawing. Note that this growth curve differs from the growth curve representing the number of bacteria.

As shown in FIG. 2, during the exponential growth phase of the colony radius, all bacteria forming the colony are able to take up nutrients from the solid medium. The colony expands as all bacteria multiply. During this period, the colony also increases in height. During the linear growth phase of the colony radius, bacteria at the center of the colony lose access to nutrients diffusing from the edges of the colony. The growth zone at the colony edge becomes constant in width. Consequently, the rate of radial growth becomes linear. The radius of the colony at which this rate of growth becomes linear is denoted as Rₗᵢₙ. When the colony grows further and reaches the saturation phase, the radius of the colony ceases to increase.

Let Rₜₕᵣₑₛₕ represent the radius of the colony at the point of appearance, as shown in FIG. 2. This Rₜₕᵣₑₛₕ indicates the limit of detectable colonies for each analysis method. Therefore, whether the appearance time is early or late depends on the performance of the analysis method. Depending on the analysis method, this appearance point may be defined as a “lag.” In an ideal sense, the lag is the point at which the colony radius becomes greater than zero, as shown on the graph. While the time of appearance can be advanced by increasing the resolution of microscopy and image analysis, it is challenging to match the appearance time with the ideal lag.

Monitoring in this embodiment is not dependent on the point of appearance of colonies, as shown in FIG. 2. The monitoring period is defined as the time during which microorganisms have already been seeded on the surface of the solid medium, but the diameter of the colonies has not yet reached the diameter of the saturation phase. During the monitoring period, the diameter of microbial colonies is preferably between 1 μm and 1 mm. For example, the colony diameter may be one of the following values: 2, 5, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, or 500 μm. The monitoring period may be set before the phase of linear growth in colony diameter on the solid medium 14, or it may be set during the linear growth phase.

### <Detection of Transmitted Light Intensity>

Returning to FIG. 1, as shown in the upper part, monitoring is performed by detecting the intensity of light transmitted through the solid medium over time. In one embodiment, monitoring is conducted by measuring the optical density over time based on the light intensity. The light intensity is measured using time-lapse imaging at the optical sensor elements 12 on the array 11. The time-lapse interval can range from 1 second to 24 hours. For example, the time-lapse interval may be one of the following values: 2, 5, 10, 20, 40, or 60 seconds; 2, 5, 10, 20, 40, or 60 minutes; or 2, 3, 6, or 12 hours. The time-lapse interval may remain constant or vary during monitoring.

In FIG. 1, light detection is performed simultaneously at all optical sensor elements 12 on the array 11 at each time-lapse interval. In other words, a frame comprising light intensity values for each culture medium coordinates is acquired. Within the frame, each culture medium coordinates can be represented as a pixel. The distribution of light intensity across the culture medium coordinates system can also be captured as an image. In another embodiment, detection may be performed at a subset of the optical sensor elements 12 on the array 11 at a given time, with other subsets being used for detection at other times.

The light detected by the optical sensor elements 12 in FIG. 1, used for monitoring, may be visible light or light of at least one of the following colors: purple, blue, light blue, green, yellow, orange, or red. In one embodiment, the wavelength of the light used for monitoring may range from 360 to 830 nm, for example, from 380 to 780 nm, or from 400 to 760 nm, and preferably from 400 to 700 nm. The wavelengths of light used may include one of the following values: 410, 420, 430, 440, 450, 460, 470, 480, 490, 500, 510, 520, 530, 540, 550, 560, 570, 580, 590, 600, 610, 620, 630, 640, 650, 660, 670, 680, or 690 nm. The colors of light used may include, for example, yellow (570 to 590 nm), orange (590 to 610 nm), or red (610 to 780 nm). These colors are particularly suitable for predicting microbial growth.

In FIG. 1, the light whose intensity is detected by the light sensor element 12 may be visible light or light of at least one of the following colors: purple, blue, light blue, green, yellow, orange, and red. The wavelength of the light detected by the light sensor element 12 may range from 360 to 830 nm, for example, from 380 to 780 nm, or from 400 to 760 nm, and preferably from 400 to 700 nm. The wavelength of the light whose intensity is detected may be one of the following values: 410, 420, 430, 440, 450, 460, 470, 480, 490, 500, 510, 520, 530, 540, 550, 560, 570, 580, 590, 600, 610, 620, 630, 640, 650, 660, 670, 680, or 690 nm. The color of the light whose intensity is detected may be, for example, at least one of the following: yellow (570 to 590 nm), orange (590 to 610 nm), or red (610 to 780 nm). These colors are suitable for predicting microbial growth.

In the upper part of FIG. 1, the light reaching the light sensor element 12 is visible light. The light reaching the light sensor element 12 may be visible light or light of at least one of the following colors: purple, blue, light blue, green, yellow, orange, and red. In one aspect, the light reaching the light sensor element 12 may have a peak wavelength ranging from 360 to 830 nm, for example, from 380 to 780 nm, or from 400 to 760 nm, and preferably from 400 to 700 nm. The peak wavelength may be one of the following values: 410, 420, 430, 440, 450, 460, 470, 480, 490, 500, 510, 520, 530, 540, 550, 560, 570, 580, 590, 600, 610, 620, 630, 640, 650, 660, 670, 680, or 690 nm. The color of the light reaching the light sensor element 12 may be at least one of the following: yellow (570 to 590 nm), orange (590 to 610 nm), or red (610 to 780 nm). These colors are suitable for predicting microbial growth.

When white light is irradiated onto the array 11, as shown in the upper part of FIG. 1, a color filter may be placed between the array 11 and the solid medium 14 to generate light of the desired wavelength. A color filter may also be provided on the light sensor element 12. Using these filters, the light sensor element 12 may detect light of at least one of the colors yellow, orange, or red, and transmit signals of the intensity of such light to the recorder 13. The recorder 13 records the intensity of at least one of the colors yellow, orange, or red. The recorder 13 also records the time at which the signal is received. In another embodiment, the array 11 may detect the intensity of light in the three primary colors (blue, green, and red; RGB) through filters provided on each light sensor element 12. The recorder 13 may record the intensity of at least the red light among the three primary colors. The recorder 13 may record the intensity of all three primary colors, and only the intensity of red light may be used to predict microbial growth.

### <Prediction of Microbial Growth>

The growth of microorganisms 16 is predicted based on the temporal decrease in the intensity of light of any of the above colors, e.g., red, at the culture medium coordinates of the solid medium 14 shown in FIG. 1. Optical density is measured over time based on the intensity of light detected.

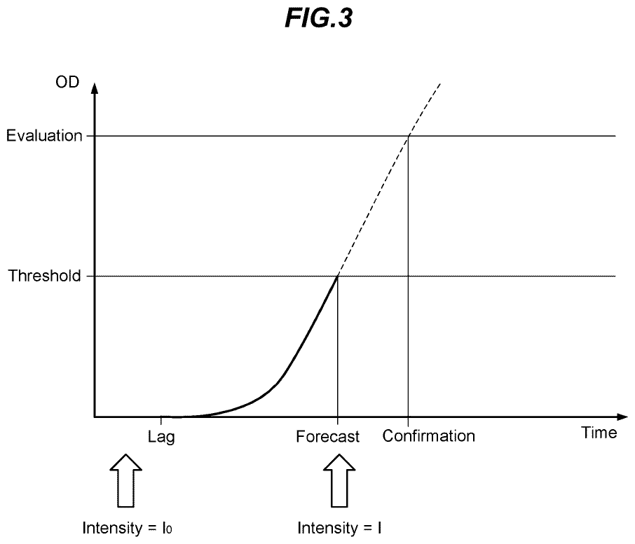

FIG. 3 illustrates the temporal change in optical density (OD) at specific culture medium coordinates. The point in time when the optical density (OD) exceeds a predetermined threshold (Threshold) is defined as the forecast time (Forecast). It is determined that a colony will subsequently form at that culture medium coordinates at the forecast time (Forecast). This process is used to predict microbial growth.

As shown in FIG. 3, at the forecast time (Forecast), the optical density (OD) has not yet reached the predetermined definitive evaluation value (Evaluation). The threshold (Threshold) is applied to the culture medium coordinates where the optical density (OD) has not reached the definitive evaluation value (Evaluation). At some point after the forecast time (Forecast) referred to as Confirmation, the optical density (OD) reaches the definitive evaluation value (Evaluation) with a certain probability. The threshold (Threshold) is defined as the optical density value that indicates the likelihood of the optical density (OD) reaching the definitive evaluation value (Evaluation).

The optical density (OD) shown in FIG. 3 is calculated using the following formula:

$$[ Optical Density ( OD ) ] = Log ( {I}_{0} / I )$$

where I is the intensity of light (Intensity) detected at a specific point in time, e.g., at the forecast time (Forecast) I₀ is the so-called blank value of light intensity (Intensity). I₀ is, for example, the average intensity of light over a predetermined number of consecutive detections, including the first detected light intensity.

### <Monitoring Before Colony Emergence>

The above embodiment predicts microbial growth. The device and method described in this embodiment can also be applied to monitoring microbial growth before the appearance of a colony.

Returning to FIG. 1, solid medium 14 is overlaid on array 11, where light sensor elements 12 are deployed. The alignment between the detection coordinates, which are the two-dimensional coordinates of the light sensor elements 12 on array 11, and the culture medium coordinates, which are the two-dimensional coordinates on solid medium 14, is maintained over time. While maintaining this alignment, the intensity of light reaching the detection coordinates from the opposite side across each culture medium coordinates is detected by each light sensor element 12 over time. The temporal changes in the intensity of visible light at the culture medium coordinates are observed.

The optical density measured over time, based on the light intensity detected over time in FIG. 1, is calculated using the following formula:

$$[ Optical Density ( OD ) ] = Log ( {I}_{0} / I )$$

where I is the light intensity detected at each time point at the detection coordinates, and I₀ is the average of the light intensity over a predetermined number of consecutive detections, including the first light intensity detected at the detection coordinates.

### EXAMPLES

### Overview

Non-Patent Literatures 1-3 analyze each frame in a time-lapse image. As explained using FIG. 2, image analysis becomes possible only after the colony has appeared. Specifically, analysis is conducted after the colony has grown to a size of several hundred micrometers.

In contrast, in Examples 1 through 3 below, as shown in FIG. 1, the light intensity obtained from the light sensor elements 12 in time-lapse imaging was converted into optical density. Each light sensor element 12 at the detection coordinates was treated as if it were an absorbance meter measuring changes in optical density during liquid culture of microorganisms. In Example 4, instead of converting light intensity into optical density, a statistical background subtraction method was applied to the temporal changes in light intensity. In Example 5, a chromogenic agent for detecting microorganisms was added to the solid medium to enable specific monitoring for biological classification. Example 6 demonstrates the acquisition of a blank value (I₀). Example 7 illustrates the summation of light intensity detected by multiple light sensor elements.

In all examples, solid medium 14 was overlaid on array 11, enabling the monitoring of microbial growth at each culture medium coordinate across a wide area of solid medium 14.

### <Array of Light Sensor Elements and Time Intervals>

A TFT image sensor was used as the array to detect light intensity.
length and breadth (Pixel size) of photodiode: 80×80 μm
number of photodiodes in lengthwise and breadthwise directions (Sensor array): 542 pixels×872 pixels
array area (Field of view): 44×70 mm
percentage of dish covered by array: Covers 55% of a 90-mm petri dish
time interval: measured every 5 minutes

### Example 1: Detection of Light Intensity and Measurement of Optical Density

Enterobacter cloacae (GTC 21793) was cultured on non-selective medium. Bacteria were isolated from a single colony on this non-selective medium. The bacteria were suspended in 400 μL of sterile PBS. A disposable loop was immersed in the suspension to collect 10 μL of the suspension on the disposable loop. This suspension was streaked onto the medium in a dish, ChromoAgar mSuper CARBA (Kanto Chemical, 72141).

In this example, a dish (container 15) seeded with microorganisms was placed on the apparatus 10 shown in FIG. 1. The microorganisms were incubated at 37° C. for 17.5 hours. Every 5 minutes, the intensity of light at a wavelength of 630 nm transmitted through the medium (solid medium 14) was measured across the array 11, producing frames that comprise the distribution of light intensity within the system of culture medium coordinates. A total of 211 frames of data were obtained.

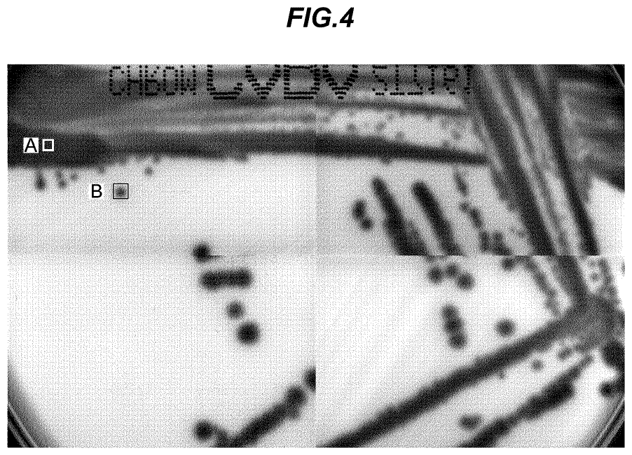

FIG. 4 shows the distribution of light intensity within the system of culture medium coordinates in the final frame, corresponding to the saturation phase. Brighter areas indicate higher light intensity. The light intensity is low at and around culture medium coordinates A. At culture medium coordinates B, the light intensity is low, but it is higher in the surrounding area.

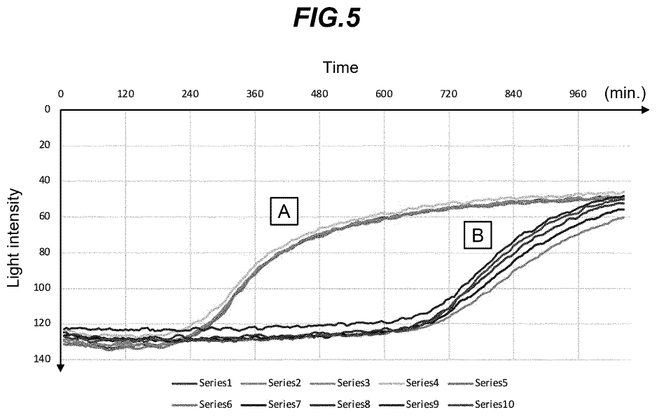

FIG. 5 illustrates the temporal changes in light intensity at five coordinates centered on culture medium coordinates A and B, respectively, as shown in FIG. 4. The vertical axis represents light intensity, which increases downward. The horizontal axis represents time (minutes). Series 1 to 5 correspond to coordinates centered on culture medium coordinates A, while Series 6 to 10 correspond to coordinates centered on culture medium coordinates B. A decrease in light intensity was observed at each of the culture medium coordinates.

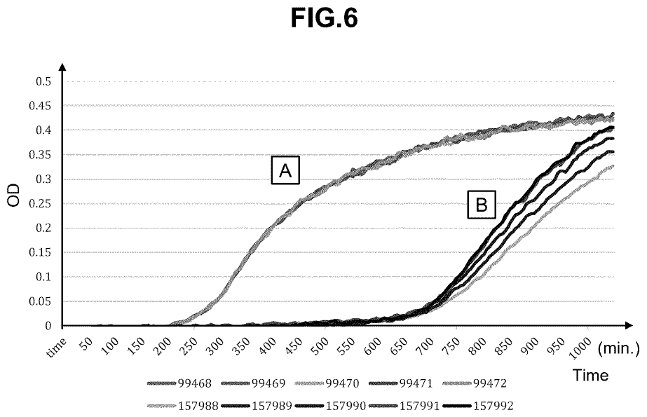

FIG. 6 shows the temporal changes in optical density (OD) at five coordinates centered on culture medium coordinates A and B, as depicted in FIG. 4. The vertical axis represents optical density, and the horizontal axis represents time. Positions of coordinates 99468 to 99472 are centered on culture medium coordinates A, while coordinates 157988 to 157992 are centered on culture medium coordinates B. An increase in optical density was observed at each culture medium coordinates.

As shown in FIGS. 5 and 6, the periods during which light intensity and optical density changed significantly differed between the culture medium coordinates A and B. This confirmed the presence of heterogeneity, where the growth rate varied among individual microorganisms of the same species that were seeded.

### Example 2: Prediction of Microbial Growth

In this example, microorganisms were detected at an early stage by identifying the rise in the growth curve, as shown in FIG. 3. In parallel with Example 1, the temporal changes in light intensity and optical density were recorded at all detection coordinates (corresponding to 472,624 pixels) on the array. Initially, predictions of microbial growth at all culture medium coordinates were made based on the temporal changes in light intensity. However, the precision of these predictions (Precision) was not high. Next, the growth of microorganisms at the position in the medium was predicted based on the temporal change in optical density shown in FIG. 6.

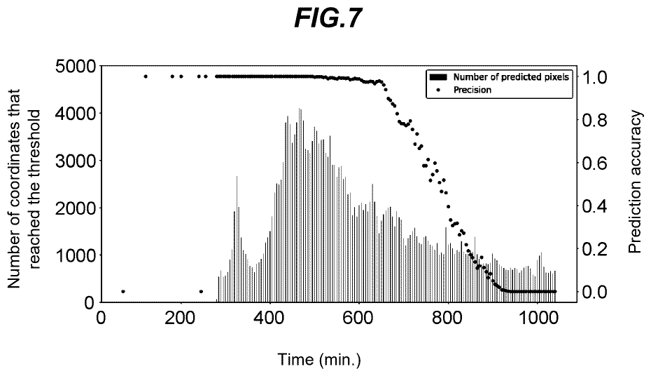

FIG. 7 shows the temporal change in the number of culture medium coordinates (Number of predicted pixels) that first reached the optical density threshold and the temporal change in the prediction accuracy (Precision). The optical density threshold was set at OD=0.05. The optical density (OD) in the final frame was set at 0.1 or higher as an indicator to confirm the prediction. After recording was completed up to the final frame, retrospective determinations of microbial growth predictions were made for each frame.

As shown in FIG. 7, which shows the graph of changes over time, it was determined that predictions with an accuracy rate of nearly 100% are possible beginning around frame of 250 minutes (frame #50). This accuracy was higher than that achieved when predicting microbial growth based on the temporal changes in light intensity. In other words, higher precision predictions were obtained from earlier frames. The decrease in prediction precision after 650 minutes was due to slower microbial growth in certain culture medium coordinates. Specifically, some culture medium coordinates where the OD reached the threshold value of 0.05 did not exceed an OD of 0.1 by the final frame.

### Example 3: Early Measurement of Zone of Inhibition Diameter

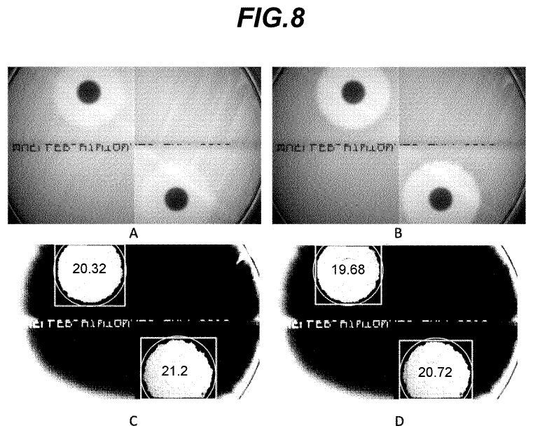

Using the prediction of microbial growth described above and the light intensity frames, the diameter of the zone of inhibition is measured early, preferably before the colony appearance. FIGS. 8A and 8B show the distribution of light intensity indicating the zone of inhibition.

First, a McFarland 0.5 bacterial suspension was prepared by suspending colonies of E. coli (NBRC 15034) cultured on non-selective medium in saline. A sterile cotton swab soaked in this suspension was slid over the surface of Mueller-Hinton II agar medium (BD, 251177) from three or more different directions at an angle of approximately 60 degrees. By soaking the sterile cotton swab with the bacterial suspension, excess liquid was absorbed into the sterile cotton swab from the bacterial suspension. The bacterial suspension was evenly applied to the medium, and the medium was left to stand for 3 to 5 minutes, thereby inoculating the microorganisms onto the medium.

Next, as shown in FIGS. 8A and 8B, agents were placed in a scattered pattern on the surface of the medium. In this example, two types of antimicrobial discs were placed on each agar medium plate inoculated with bacteria. The upper-left disc contained amoxicillin/clavulanic acid, while the lower-right disc contained ampicillin/sulbactam.

Subsequently, a frame comprising the system of the culture medium coordinates and the light intensity at each culture medium coordinate was acquired over time using an array of optical sensor elements. In this example, the culture was incubated at 37° C. for 25 hours while detecting light intensity over time. A total of 300 frames of data were acquired by performing detection every 5 minutes. FIG. 8A represents a frame after 8.3 hours, while FIG. 8B represents a frame during the saturation phase after 25 hours.

Next, within each frame, the contour of the set of coordinates where the optical density does not exceed the threshold was extracted. The diameter of the set whose contour was determined to be circular was also extracted. In this example, the diameter was extracted by treating the distribution of light intensity within the frame as an image.

The procedure is as follows:
Convert the light intensity values of all coordinates in the initial frame (Frame 1) to 255 (white, 8-bit grayscale).
From the second frame (Frame 2) to the 300th frame, predict microbial growth using the optical density method described in Example 2. Convert the light intensity at the culture medium coordinates (pixels) where growth is predicted to 0 (black, 8-bit grayscale).
Extract the black-and-white boundary as a closed ring from the binarized frame generated by the above process.
Calculate the diameter of the extracted rings.

FIG. 8C illustrates the area surrounded by pixels predicted to proliferate based on the OD in Frame A, representing the zone of inhibition and its diameter (mm). FIG. 8D illustrates the area surrounded by pixels predicted to proliferate based on the OD in Frame B, representing the zone of inhibition and its diameter (mm). As shown in these figures, the diameter of the zone of inhibition could be measured in approximately one-third of the time required to reach the saturation phase.

### Example 4: Prediction Using Statistical Background Subtraction Method for Frames

In this example, frames were analyzed without using optical density. Specifically, the temporal changes in the distribution of light intensity obtained in Example 2 were analyzed as two-dimensional data comprising intensity axis and a time axis. The statistical background subtraction method, commonly used in video analysis, was employed for this analysis. This method enables robust detection against noise generated by optical sensor elements and other components.

First, microorganisms at high concentration (clinical) or low concentration (single colony) were seeded and cultured on ChromoAgar mSuper CARBA growth medium (Kanto Chemical). High concentration (clinical) refers to seeding a sufficient number of bacteria to generate zonal colonies during the saturation phase. Low concentration (single colony) refers to seeding a smaller number of bacteria, resulting in sporadic colonies during the saturation phase. Three species of microorganisms were used: E. cloacae, E. coli, and K. pneumoniae.

Time-lapse videos were created by detecting the distribution of light intensity over time using a TFT image sensor. Three repeated experiments were conducted for each microbial species. Statistical background subtraction was performed using a software, OpenCV algorithm, cv2.bgsegm.createBackgroundSubtractorKNN( ).

As mentioned above, the prediction of microbial growth using optical density employed the light intensity during the lag phase as the blank value (I₀). In the statistical background subtraction method, the background light intensity is calculated from the light intensity of a specified number of consecutive detections, including the most recently detected light intensity. For example, the number of detections is specified using the history parameter that can be adjusted in cv2.bgsegm.createBackgroundSubtractorKNN( ). This background is updated each time a new frame is acquired.

When the intensity of the detected light decreases over time and separates from the background light intensity, it is determined that a colony will subsequently form at that medium coordinate. The separation from the background light intensity is calculated based on a predetermined threshold value. For example, the threshold can be specified using the varThreshold parameter, which is adjustable in the cv2.bgsegm.createBackgroundSubtractorKNN( ) constructor.

Table 1 shows the time (detection time) at which predictions with a prediction accuracy (Precision) of 1.0 became possible. “n” indicates the first, second, and third trials, respectively.

To calculate the prediction accuracy (Precision), correct annotations were assigned to the culture medium coordinates (pixels) where microorganisms were growing in the final frame. The presence of microbial growth at each culture medium coordinates (pixel) in the final frame was determined using a predetermined light intensity threshold. This threshold value may be determined with reference to colony identification results obtained by the human eye. The ratio of culture medium coordinates with the correct annotations among those that predicted microbial growth is defined as “precision.” Precision can be set to any value less than 1.0.

For comparison, the same threshold value was used in the simple background subtraction method, and the detection time was calculated with Precision set to 1.0. The intensity of light for a series of consecutive frames, including the first, was used as the fixed background. In other words, the light intensity during the lag period was used as the fixed background.

In all datasets, the statistical background subtraction method demonstrated faster detection times. Precision was set to 1.0 for all datasets, indicating that this method correctly predicted the culture medium coordinates (pixels) where colonies appeared, rather than detecting noise.

TABLE 1
A comparison of detection times using statistical and
simple background subtraction (E. cloacae)

| | E. cloacae | | | | | |
|---|---|---|---|---|---|---|
| | high conc. (clinical) | | | low conc. (single colony) | | |
| | detection time (h) | | | | | |
| | n = 1 | n = 2 | n = 3 | n = 1 | n = 2 | n = 3 |
| simple | 4.4 | 4.9 | 5.6 | 8.6 | 8.9 | 9.7 |
| background | | | | | | |
| subtraction | | | | | | |
| statistical | 4.3 | 4.7 | 5.2 | 8.4 | 8.7 | 9.4 |
| background | | | | | | |
| subtraction | | | | | | |

A comparison of detection times using statistical and
simple background subtraction (E. coli)

| | E. coli | | | | | |
|---|---|---|---|---|---|---|
| | high conc. (clinical) | | | low conc. (single colony) | | |
| | detection time (h) | | | | | |
| | n = 1 | n = 2 | n = 3 | n = 1 | n = 2 | n = 3 |
| simple | 4.8 | 4.2 | 5.2 | 7.3 | 8.2 | 8.1 |
| background | | | | | | |
| subtraction | | | | | | |
| statistical | 4.5 | 4.2 | 5 | 7 | 8.1 | 7.8 |
| background | | | | | | |
| subtraction | | | | | | |

A comparison of detection times using statistical and
simple background subtraction (K. pneumoniae)

| | K. pneumoniae | | | | | |
|---|---|---|---|---|---|---|
| | high conc. (clinical) | | | low conc. (single colony) | | |
| | detection time (h) | | | | | |
| | n = 1 | n = 2 | n = 3 | n = 1 | n = 2 | n = 3 |
| simple | 4.2 | 4.4 | 4.6 | 9 | 8.5 | 8.2 |
| background | | | | | | |
| subtraction | | | | | | |
| statistical | 4.1 | 4.2 | 4.3 | 8.7 | 8.3 | 8 |
| background | | | | | | |
| subtraction | | | | | | |

### Example 5: Analysis Using Color Space

This example demonstrates that various microorganisms, including pathogenic bacteria, can be detected and identified at an early stage. The method and apparatus described in this example can help address important public health challenges. Conventional methods of growing microorganisms in specimens on solid media typically require overnight incubation to obtain definitive results. Validation of this example shows that the thin-film transistor (TFT) image sensor used in this study exhibits high performance for the early detection of microbial colonies on solid media. The device used in this example detects transmitted light.

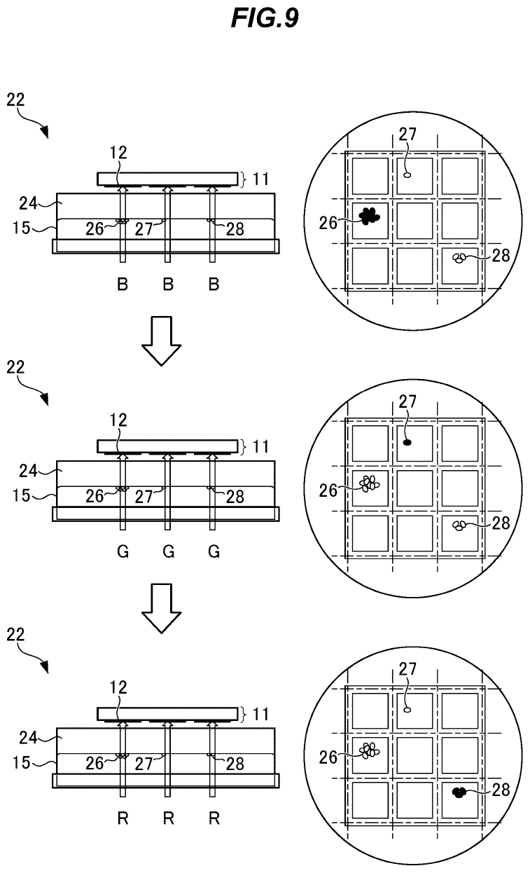

FIG. 9 illustrates the cycle of the three primary colors of light irradiating the solid medium 24 on device 22. In this example, blue (B), green (G), and red (R) light are sequentially irradiated onto the solid medium 24. This order is one example and can be modified according to the design of the device. The blue (B), green (G), and red (R) lights are generated independently by LED lights, with separate timing for each.

In FIG. 9, the solid medium 24 contains enzyme substrates. When bacterial species 26, 27, and 28 are actively growing, each species produces enzymes characteristic of its biological classification. These substrates undergo chemical reactions, causing coloration at the culture medium coordinates where each bacterial species is growing in the solid medium 24. The coloration is detected at the detection coordinates on array 11, which is positioned opposite the light source across the solid medium 24. By cycling the irradiation of the three primary colors, the light transmitted through the solid medium 24 is used to observe the coloration caused by the growth of bacterial species 26, 27, and 28. This enables the identification of the biological classifications of these bacterial species growing on the solid medium 24.

In the example shown in FIG. 9, bacterial species 26 absorbs blue (B) light, bacterial species 27 absorbs green (G) light, and bacterial species 28 absorbs red (R) light. Each bacterial species transmits light of other colors. Visually, colonies of bacterial species 26 may appear yellow, colonies of bacterial species 27 may appear red or magenta, and colonies of bacterial species 28 may appear light blue or cyan. However, at the time of detection by device 22, these colonies may be too small to be visible to the naked eye.

In FIG. 9, device 22 is a TFT image sensor comprising an unshown control printed circuit board, array 11 with light sensor elements 12, and an unshown planar illumination module. Array 11 comprises 542×872 pixel light sensor elements 12. The size of each light sensor element 12 is 80 μm in lengthwise and breadthwise direction. No lens is installed on the light sensor elements 12. No color filter is installed on the light sensor elements 12. Device 22 can capture changes on the solid medium 24 at a 1:1 magnification. The field of view (FOV) of the TFT image sensor is 44×70 mm, covering approximately 55% of the solid medium on a 90-mm petri dish. The light sensor elements on the array measure the intensity of light passing through the solid medium.

In FIG. 9, light was irradiated onto the solid medium 24 every 5 minutes. During each irradiation cycle, blue (B), green (G), and red (R) light passed through the solid medium 24 sequentially at separate timings. Based on the detected light, pseudo-color images were generated. When generating the pseudo-color images, the intensity of the detected blue (B), green (G), and red (R) light may be corrected using scaling factors that approximate human visual perception.

In this example, CHROMAGAR mSuper CARBA agar medium was used as the solid medium 24 shown in FIG. 9. Enterobacter cloacae, Escherichia coli, and Klebsiella pneumoniae bacterial species were inoculated onto the agar medium. These bacterial species grown on the agar medium were used to evaluate the detection capability of device 22. Color images of bacterial colonies grown on agar plates were automatically collected at 5-minute intervals.

The optical densities OD-R, OD-G, and OD-B for each detection coordinates (pixel) and each time point (frame) were calculated based on the transmitted light intensity when irradiated by the respective red (R), green (G), and blue (B) light sources, using the previously described formula. Here, the pixel coordinates may include all pixels within the field of view (FOV). In each frame, the outline of the colony can be extracted, and the center of the colony may be treated as the representative detection coordinate.

The obtained optical density values in the coordinates of RGB color space {OD-R, OD-G, OD-B} may be converted to optical density values in the coordinates of HSV color space {OD-H, OD-S, OD-V}. Alternatively, the coordinates of the RGB color space values {OD-R, OD-G, OD-B} may be converted to optical density values in the coordinates of HSL color space {OD-H, OD-S, OD-L}.

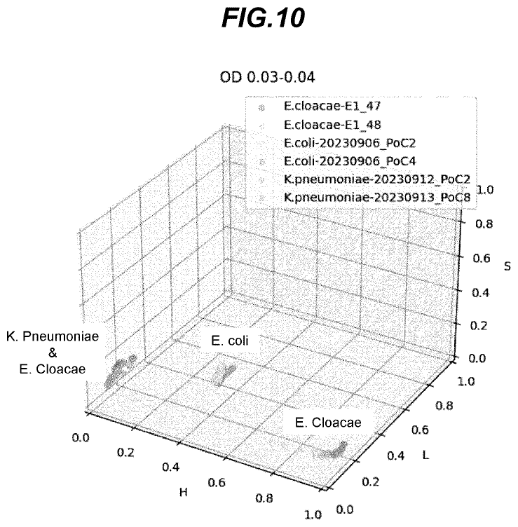

As shown in FIG. 10, this example involved plotting the values of the coordinates {OD-H, OD-S, OD-L} in HSL color space. The values OD-H, OD-S, and OD-L for the pixel and frame of interest were plotted in a three-dimensional space with H, S, and L as axes. The plot position may vary depending on the abundance of microorganisms. In such cases, microbial growth can be detected based on changes in the plot position within the color space coordinates.

As shown in FIG. 10, the values of each axis (OD) in the color space were plotted within the range of 0.03 to 0.04. The plot groups for E. coli (green in the color image), E. cloacae (pink in the color image), and K. pneumoniae (blue in the color image) were separated in the center, lower-right, and lower-left regions, respectively. A pink plot was observed in the part of the lower-left region. This is attributed to certain strains of E. cloacae producing enzymes that change the substrate to blue, depending on the taxonomy of E. cloacae.

As shown in FIG. 10, microorganisms can be separated by biological classification within the color space. Furthermore, using k-means clustering (k=3), it was found that some or all microorganisms can be identified based on their color space coordinates values {OD-H, OD-S, OD-L}. Additionally, by plotting time-series changes, it was discovered that biological classification can also be identified through the temporal variation of plot positions within the color space.

In this example, a dataset comprising reference data for known bacteria by biological classification was used as a reference. By comparing the histograms of query data from unknown bacteria with the reference data, the label of the reference data with the highest similarity or correlation coefficient can be used as the inferred label for the test data.

Clustering methods other than k-means clustering can also be used as analysis methods. In addition to clustering, rule-based analysis methods for multidimensional data, such as principal component analysis and multidimensional principal component analysis, can be applied. Furthermore, analysis methods using machine learning can also be employed in addition to the rule-based analysis methods.

In the machine learning-based analysis method, RGB images and HSV images are first obtained based on the optical density measured for each detection coordinate. For each channel, the pixel value for each detection coordinates and its frequency within the image are calculated. These values are used as features in the data table. Based on these features, a random forest classifier is employed to identify the biological classification for each detection coordinate.

In another machine learning-based analysis method, histograms are created for each channel of the HSV image by extracting pixel values and their frequencies for each channel. These histograms are then integrated into a new three-channel image, resulting in a color image of the histogram itself. This color image is used to create an image classification model for identifying the biological classification of microorganisms.

In tests conducted by the inventors, both the machine learning-based analysis method and the rule-based analysis method achieved high accuracy, exceeding 0.95.

### Example 6: Obtaining a Blank Value (I₀)

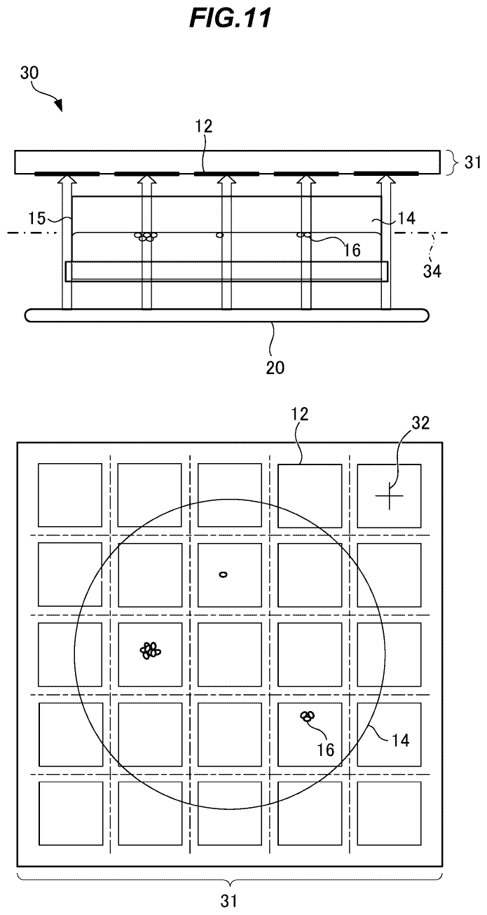

FIG. 11 shows a side view of apparatus 30. Apparatus 30 is equivalent to apparatus 10 shown in FIG. 1 but differs in the following aspects. Apparatus 30 is equipped with array 31. Array 31 is similar to array 11 shown in FIG. 1 but differs in that it is larger than the solid medium 14 and its container 15.

The lower part of FIG. 11 shows a bottom view of solid medium 14 and array 31. As mentioned above, obtaining a blank value (I₀) is necessary for calculating optical density. In this example, the detection coordinates 32 for reference are aligned with two-dimensional coordinates that are known in advance not to support microbial growth. In one example, such two-dimensional coordinates are located on an extended plane 34 of the culture medium coordinates plane. In this example, light is detected at reference detection coordinates 32, where the solid medium 14 is not overlaid. The detected intensity is taken as the blank value (I₀).

The method for obtaining the blank value (I₀) in this example is applicable to the embodiments described above, including the prediction of colony appearance, monitoring before colony appearance, and microorganism identification.

### Example 7: Summation of Detected Light Intensities

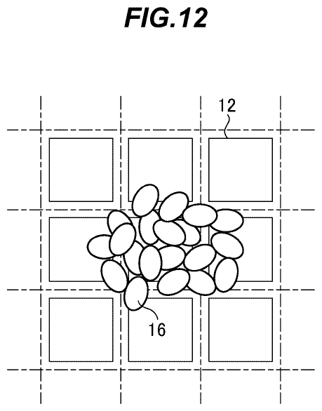

FIG. 12 shows a bottom view of the light sensor elements 12 and a population of microorganisms 16. In this example, the size of the light sensor elements 12 is small relative to the size of the microorganism population. The light intensity detected by multiple adjacent light sensor elements 12 are binned. It means that microbial growth is predicted based on the summed light intensities. In another example, optical density is calculated based on the summed values. In yet another example, microbial monitoring is performed based on the summed values. The method described in this example can also be applied when the optical sensor elements are large relative to the size of the microorganism 16 population.

The invention is not limited to the above descriptions and can be modified as necessary without departing from its scope. This application claims the benefit of foreign priority to Japanese Patent Application No. 2023-086138, filed on May 25, 2023, which is incorporated by reference in its entirety.

### DESCRIPTION OF REFERENCE NUMERALS

10: device, 11: array, 12: light sensor element, 13: recorder, 14: solid medium, 15: container, 16: microorganism, 18: stage, 19: fixed member, 20: surface light source, 22: device, 24: solid medium, 26: bacterial species, 27: bacterial species, 28: bacterial species, 30: device, 31: array, 32: detection coordinate, 34: extension plane

1. A method for monitoring growth of microorganisms, comprising,
in a state where a solid medium is superimposed on an array in which optical sensor elements larger than microorganisms are deployed when viewed in plan view, maintaining alignment over time between two-dimensional coordinates of the optical sensor elements on the array, hereinafter referred to as detection coordinates, and two-dimensional coordinates on the solid medium, hereinafter referred to as culture medium coordinates,
detecting while maintaining the alignment, light intensity arriving at the detection coordinates from the opposite side across each the culture medium coordinates by each of the optical sensor elements over time, and
observing a change over time in intensity of visible light at the culture medium coordinates.

2. The method according to claim 1, comprising predicting microbial growth based on a decrease in intensity of visible light over time.

3. The method according to claim 2, comprising
predicting the growth of the microorganisms by determining that a colony will subsequently occur at the culture medium coordinates at a point in time when the optical density measured over time based on intensity of light detected over time exceeds a predetermined threshold value, hereinafter referred to as a prediction time, wherein
the threshold value is an optical density value indicating that the optical density will reach a predetermined definitive evaluation value with a predetermined probability at any subsequent time at the culture medium coordinates where the optical density did not reach the predetermined definitive evaluation value at the prediction time.

4. The method according to claim 3, comprising calculating an optical density based on the following equation,
$$[ Optical Density ( OD ) ] = Log ( {I}_{0} / I )$$
wherein I is intensity of light detected at each time point at the detection coordinate, and
I₀ is an average of light intensity for a given number of consecutive detections, including first light intensity detected at the detection coordinates, or I₀ is reference light intensity detected at each time point at reference detection coordinates aligned with two-dimensional coordinates on an extension of a plane of the culture medium coordinates that is known in advance not to grow microorganisms.

5. The method according to claim 3, comprising,
dispersing an agent on a surface of the solid medium,
obtaining frames comprising the culture medium coordinates and light intensity on the culture medium coordinates over time at the array,
extracting a contour of a set of coordinates in the frame for which the optical density does not exceed the threshold value, and
extracting a diameter of the set in which the contour is determined to be circular.

6. The method according to claim 2, comprising determining that a colony subsequently arises on the culture medium coordinates at a point in time when intensity of detected light over time decreases and separates from intensity of background light, using a statistical background differencing method, wherein
background light intensity is calculated based on light intensity for the latest predetermined number of consecutive detections, including the most recently detected light intensity, and
the separation from the background light intensity is calculated based on a predetermined threshold value.

7. The method according to claim 1, comprising predicting microbial growth based on a decrease over time of combined intensity of light detected by a plurality of adjacent optical sensor elements.

8. The method according to claim 1, comprising calculating an optical density measured over time based on light intensity detected over time using the following equation,
$$[ Optical Density ( OD ) ] = Log ( {I}_{0} / I )$$
where I is light intensity detected at each time point at the detection coordinates, and
I₀ is an average of light intensity for a given number of consecutive detections, including the first light intensity detected at the detection coordinates, or I₀ is a reference light intensity detected at each time point at reference detection coordinates aligned with two-dimensional coordinates on the extension of the plane of the culture medium coordinates that is known in advance not to grow microorganisms.

9. The method according to claim 8, wherein
I₀ is light intensity detected at each time point at the reference detection coordinates, and
the reference detection coordinates do not have the solid medium overlaid on them.

10. The method according to claim 8, wherein the optical density measured over time is calculated based on the sum of light intensity detected by a plurality of adjacent optical sensor elements.

11. The method according to claim 1, wherein
the solid culture medium contains an indicator that specifically reacts and changes color in response to the activity of microorganisms belonging to a particular biological classification, and
the visible light is within a wavelength range that is absorbed by the colored indicator.

12. The method according to claim 11, comprising,
further observing over time, intensity of light within a wavelength range that is not absorbed by the colored indicator, and
calculating color space coordinates at the culture medium coordinates.

13. The method according to claim 12, wherein
the solid culture medium contains multiple types of indicators,
each of the indicators specifically reacts to growth of microorganisms belonging to different biological classifications, with each indicator producing a distinct color, and
the biological classification of microorganisms at the culture medium coordinates is identified by calculating the color space coordinates at the culture medium coordinates.

14. The method according to claim 13, wherein
the light reaching the detection coordinates is divided into blue, green, and red light, and
timing each color of light separately to ensure arrival at the detection coordinates when detected by the optical sensor elements.

15. The method according to claim 13,
identifying biological classifications using at least one of the following methods, k-means clustering and other clustering-based analysis methods; principal component analysis, multidimensional principal component analysis and other multidimensional data analysis methods; and rule-based analysis methods; and analysis methods using machine learning.

16. The method according to claim 1, wherein a scale of the culture medium coordinates is equal to a scale of the detection coordinates.

17. The method according to claim 1, wherein
the optical sensor element is larger than the microorganism when viewed in plan view, or
the optical sensor element comprises a microlens and a photodetector element, wherein the microlens is larger than the microorganism and the photodetector element is smaller than the microorganism when viewed in plan view.

18. The method according to claim 1, wherein, a wavelength of the visible light is within the range of 400 to 700 nm.

19. An apparatus for monitoring growth of microorganisms, comprising:
a stage for placing a container that holds a solid medium;
an array with arranged optical sensor elements, each of which is larger than the microorganism when viewed in plan view, and which is fixed in position relative to the stage so as to overlap the solid medium;
a surface light source provided on the opposite side of the array across the stage;
and a recorder configured to receive, in real time, signals of intensity of light from the surface light source detected by the optical sensor elements, and to record intensity of visible light of the light and the time the signal was received.

20. The apparatus according to claim 19, wherein
the stage is a light-transmissive cover fixed to the surface light source, and
the container is placed on the stage.
